SEQUENCE LISTING (75 sequences)

SEQ ID NO: 1 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tcagtgtgttaatcttacatt

SEQ ID NO: 2 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
taatcttacaaccagaacttt

SEQ ID NO: 3 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tttcacacgtggtgtttattt

SEQ ID NO: 4 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgttacttggttccatgcttt

SEQ ID NO: 5 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
caatggtactaagaggttttt

SEQ ID NO: 6 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
tgtaagattaacacactgatt

SEQ ID NO: 7 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agttctggttgtaagattatt

SEQ ID NO: 8 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
ataaacaccacgtgtgaaatt

SEQ ID NO: 9 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
agcatggaaccaagtaacatt

SEQ ID NO: 10 - DNA, 21 bp, synthetic construct
  misc_feature 1..21: note = Description of Artificial Sequence: Synthetic oligonucleotide
  source 1..21: mol_type = other DNA
  misc_feature 1..19: note = RNA
  misc_feature 20..21: note = DNA
aaacctcttagtaccattgtt

SEQ ID NO: 11 - RNA, 19 nt, synthetic construct
  misc_feature 1..19: note = Description of Artificial Sequence: Synthetic oligonucleotide
  source 1..19: mol_type = other RNA
gaggactcttggactctca

SEQ ID NO: 12 - RNA, 19 nt, synthetic construct
  misc_feature 1..19: note = Description of Artificial Sequence: Synthetic oligonucleotide
  source 1..19: mol_type = other RNA
tgtcaacgtccgaccttga

SEQ ID NO: 13 - RNA, 19 nt, synthetic construct
  misc_feature 1..19: note = Description of Artificial Sequence: Synthetic oligonucleotide
  source 1..19: mol_type = other RNA
cgtccgaccttgaggcata

SEQ ID NO: 14 - RNA, 19 nt, synthetic construct
  misc_feature 1..19: note = Description of Artificial Sequence: Synthetic oligonucleotide
  source 1..19: mol_type = other RNA
tgatctttgtactaggagg

SEQ ID NO: 15 - RNA, 19 nt, synthetic construct
  misc_feature 1..19: note = Description of Artificial Sequence: Synthetic oligonucleotide
  source 1..19: mol_type = other RNA
attggtctgttcaccagca

SEQ ID NO: 16 - RNA, 19 nt, synthetic construct
  misc_feature 1..19: note = Description of Artificial Sequence: Synthetic oligonucleotide
  source 1..19: mol_type = other RNA
gaacatggacttgtatatt

SEQ ID NO: 17 - RNA, 19 nt, synthetic construct
  misc_feature 1..19: note = Description of Artificial Sequence: Synthetic oligonucleotide
  source 1..19: mol_type = other RNA
ggacgagaaagatcattga

SEQ ID NO: 18 - RNA, 19 nt, synthetic construct
  misc_feature 1..19: note = Description of Artificial Sequence: Synthetic oligonucleotide
  source 1..19: mol_type = other RNA
ggtaggatcagccctcatt

SEQ ID NO: 19 - RNA, 21 nt, synthetic construct
  misc_feature 1..21: note = Description of Artificial Sequence: Synthetic oligonucleotide
  source 1..21: mol_type = other RNA
cccgagggcgaggatgagaaa

SEQ ID NO: 20 - RNA, 19 nt, synthetic construct
  misc_feature 1..19: note = Description of Artificial Sequence: Synthetic oligonucleotide
  source 1..19: mol_type = other RNA
ggaaactgctgctgtgtac

SEQ ID NO: 21 - RNA, 20 nt, synthetic construct
  misc_feature 1..20: note = Description of Artificial Sequence: Synthetic oligonucleotide
  source 1..20: mol_type = other RNA
ggtacgactctggcattgag

SEQ ID NO: 22 - RNA, 19 nt, synthetic construct
  misc_feature 1..19: note = Description of Artificial Sequence: Synthetic oligonucleotide
  source 1..19: mol_type = other RNA
gaattgctgcttcggaatg

SEQ ID NO: 23 - RNA, 18 nt, synthetic construct
  misc_feature 1..18: note = Description of Artificial Sequence: Synthetic oligonucleotide
  source 1..18: mol_type = other RNA
gaagggaagtttcagtaa

SEQ ID NO: 24 - protein, 20 aa, synthetic construct
  REGION 1..20: note = Description of Artificial Sequence: Synthetic peptide
  REGION 1..20: note = This sequence is one part of a branched amino acid sequence
KHKHHKHHKHHKHHKHHKHK

SEQ ID NO: 25 - protein, 17 aa, synthetic construct
  REGION 1..17: note = Description of Artificial Sequence: Synthetic peptide
  REGION 1..17: note = This sequence is one part of a branched amino acid sequence
KHHHKHHHKHHHKHHHK

SEQ ID NO: 26 - protein, 18 aa, synthetic construct
  REGION 1..18: note = Description of Artificial Sequence: Synthetic peptide
KHHHKHHHKHHHHKHHHK

SEQ ID NO: 27 - protein, 18 aa, synthetic construct
  REGION 1..18: note = Description of Artificial Sequence: Synthetic peptide
  SITE 1: note = MOD_RES - D-lysine
  SITE 5: note = MOD_RES - D-lysine
  SITE 9: note = MOD_RES - D-lysine
  SITE 14: note = MOD_RES - D-lysine
  SITE 18: note = MOD_RES - D-lysine
KHHHKHHHKHHHHKHHHK

SEQ ID NO: 28 - protein, 19 aa, synthetic construct
  REGION 1..19: note = Description of Artificial Sequence: Synthetic peptide
HKHHHKHHHKHHHHKHHHK

SEQ ID NO: 29 - protein, 20 aa, synthetic construct
  REGION 1..20: note = Description of Artificial Sequence: Synthetic peptide
HHKHHHKHHHKHHHHKHHHK

SEQ ID NO: 30 - protein, 21 aa, synthetic construct
  REGION 1..21: note = Description of Artificial Sequence: Synthetic peptide
KHHHHKHHHHKHHHHKHHHHK

SEQ ID NO: 31 - protein, 18 aa, synthetic construct
  REGION 1..18: note = Description of Artificial Sequence: Synthetic peptide
KHHHKHHHKHHHKHHHHK

SEQ ID NO: 32 - protein, 18 aa, synthetic construct
  REGION 1..18: note = Description of Artificial Sequence: Synthetic peptide
KHHHKHHHHKHHHKHHHK

SEQ ID NO: 33 - protein, 19 aa, synthetic construct
  REGION 1..19: note = Description of Artificial Sequence: Synthetic peptide
KHHHKHHHHKHHHKHHHHK

SEQ ID NO: 34 - protein, 20 aa, synthetic construct
  REGION 1..20: note = Description of Artificial Sequence: Synthetic peptide
KHKHHKHHKHHKHHKHHKHK

SEQ ID NO: 35 - protein, 18 aa, synthetic construct
  REGION 1..18: note = Description of Artificial Sequence: Synthetic peptide
KHHHKHHHKHHHHKHHHK

SEQ ID NO: 36 - protein, 18 aa, synthetic construct
  REGION 1..18: note = Description of Artificial Sequence: Synthetic peptide
  SITE 1: note = MOD_RES - D-lysine
  SITE 5: note = MOD_RES - D-lysine
  SITE 9: note = MOD_RES - D-lysine
  SITE 14: note = MOD_RES - D-lysine
  SITE 18: note = MOD_RES - D-lysine
KHHHKHHHKHHHHKHHHK

SEQ ID NO: 37 - protein, 19 aa, synthetic construct
  REGION 1..19: note = Description of Artificial Sequence: Synthetic peptide
HKHHHKHHHKHHHHKHHHK

SEQ ID NO: 38 - protein, 20 aa, synthetic construct
  REGION 1..20: note = Description of Artificial Sequence: Synthetic peptide
HHKHHHKHHHKHHHHKHHHK

SEQ ID NO: 39 - protein, 21 aa, synthetic construct
  REGION 1..21: note = Description of Artificial Sequence: Synthetic peptide
KHHHHKHHHHKHHHHKHHHHK

SEQ ID NO: 40 - protein, 18 aa, synthetic construct
  REGION 1..18: note = Description of Artificial Sequence: Synthetic peptide
KHHHKHHHKHHHKHHHHK

SEQ ID NO: 41 - protein, 18 aa, synthetic construct
  REGION 1..18: note = Description of Artificial Sequence: Synthetic peptide
KHHHKHHHHKHHHKHHHK

SEQ ID NO: 42 - protein, 19 aa, synthetic construct
  REGION 1..19: note = Description of Artificial Sequence: Synthetic peptide
KHHHKHHHHKHHHKHHHHK

SEQ ID NO: 43 - protein, 9 aa, synthetic construct
  REGION 1..9: note = Description of Artificial Sequence: Synthetic peptide
  REGION 1..9: note = This sequence is one part of a branched amino acid sequence
HHHHNHHHH

SEQ ID NO: 44 - protein, 19 aa, synthetic construct
  REGION 1..19: note = Description of Artificial Sequence: Synthetic peptide
HHHKHHHKHHHKHHHKHHH

SEQ ID NO: 45 - protein, 4 aa, synthetic construct
  REGION 1..4: note = Description of Artificial Sequence: Synthetic peptide
HHHK

SEQ ID NO: 46 - protein, 5 aa, synthetic construct
  REGION 1..5: note = Description of Artificial Sequence: Synthetic peptide
HHKHH

SEQ ID NO: 47 - protein, 42 aa, synthetic construct
  REGION 1..42: note = Description of Artificial Sequence: Synthetic polypeptide
KHHHKHHHKHHHKHHHHHHKHHHKHHHKHHHKHHHHNHHHHH

SEQ ID NO: 48 - protein, 45 aa, synthetic construct
  REGION 1..45: note = Description of Artificial Sequence: Synthetic polypeptide
KHHHKHHHKHHHKHHHHHHKHHHKHHHKHHHKHHHHNHHHHHRGD

SEQ ID NO: 49 - protein, 33 aa, synthetic construct
  REGION 1..33: note = Description of Artificial Sequence: Synthetic polypeptide
HHHKHHHKHHHHHHKHHHKHHHKHHHHNHHHHH

SEQ ID NO: 50 - protein, 34 aa, synthetic construct
  REGION 1..34: note = Description of Artificial Sequence: Synthetic polypeptide
KHHHKHHHKHHHHHHKHHHKHHHKHHHHNHHHHH

SEQ ID NO: 51 - protein, 15 aa, synthetic construct
  REGION 1..15: note = Description of Artificial Sequence: Synthetic peptide
  REGION 1..15: note = This sequence is one part of a branched amino acid sequence
HHHKHHHKHHHKHHH

SEQ ID NO: 52 - protein, 11 aa, synthetic construct
  REGION 1..11: note = Description of Artificial Sequence: Synthetic peptide
HHHKHHHKHHH

SEQ ID NO: 53 - protein, 17 aa, synthetic construct
  REGION 1..17: note = Description of Artificial Sequence: Synthetic peptide
KHHHKHHHKHHHKHHHK

SEQ ID NO: 54 - protein, 20 aa, synthetic construct
  REGION 1..20: note = Description of Artificial Sequence: Synthetic peptide
KHKHHKHHKHHKHHKHHKHK

SEQ ID NO: 55 - protein, 19 aa, synthetic construct
  REGION 1..19: note = Description of Artificial Sequence: Synthetic peptide
  REGION 1..19: note = This sequence is one part of a branched amino acid sequence
KHKHKHKHKHKHKHKHKHK

SEQ ID NO: 56 - protein, 16 aa, synthetic construct
  REGION 1..16: note = Description of Artificial Sequence: Synthetic peptide
  REGION 1..16: note = This sequence is one part of a branched amino acid sequence
HHHKHHHKHHHKHHHK

SEQ ID NO: 57 - protein, 12 aa, synthetic construct
  REGION 1..12: note = Description of Artificial Sequence: Synthetic peptide
  REGION 1..12: note = This sequence is one part of a branched amino acid sequence
HHHKHHHKHHHK

SEQ ID NO: 58 - protein, 42 aa, synthetic construct; identical to SEQ ID NO: 47
  REGION 1..42: note = Description of Artificial Sequence: Synthetic polypeptide

SEQ ID NO: 59 - protein, 33 aa, synthetic construct; identical to SEQ ID NO: 49
  REGION 1..33: note = Description of Artificial Sequence: Synthetic polypeptide

SEQ ID NO: 60 - protein, 34 aa, synthetic construct; identical to SEQ ID NO: 50
  REGION 1..34: note = Description of Artificial Sequence: Synthetic polypeptide

SEQ ID NO: 61 - protein, 17 aa, synthetic construct
  REGION 1..17: note = Description of Artificial Sequence: Synthetic peptide
KHHHKHHHKHHHKHHHK

SEQ ID NO: 62 - protein, 28 aa, synthetic construct
  SITE 16: note = Side chain of lysine linked via an amide linkage to another sequence
  SITE 26: note = Side chain of lysine linked via an amide linkage to another sequence
  SITE 27: note = Side chain of lysine linked via an amide linkage to another sequence
  SITE 28: note = Side chain of lysine linked via an amide linkage to another sequence
  SITE 28: note = Lysine carboxamide
HHHKHHHKHHHKHHHKHHHHNHHHHKKK

SEQ ID NO: 63 - protein, 25 aa, synthetic construct
  SITE 13: note = Side chain of lysine linked via an amide linkage to another sequence
  SITE 23: note = Side chain of lysine linked via an amide linkage to another sequence
  SITE 24: note = Side chain of lysine linked via an amide linkage to another sequence
  SITE 25: note = Side chain of lysine linked via an amide linkage to another sequence
  SITE 25: note = Lysine carboxamide
HHHKHHHKHHHKKHHHHNHHHHKKK

SEQ ID NO: 64 - protein, 20 aa, synthetic construct
  SITE 18: note = Side chain of lysine linked via an amide linkage to another sequence
  SITE 19: note = Side chain of lysine linked via an amide linkage to another sequence
  SITE 20: note = Side chain of lysine linked via an amide linkage to another sequence
  SITE 20: note = Lysine carboxamide
KHHHKHHHKHHHKHHHKKKK

SEQ ID NO: 65 - protein, 23 aa, synthetic construct
  SITE 21: note = Side chain of lysine linked via an amide linkage to another sequence
  SITE 22: note = Side chain of lysine linked via an amide linkage to another sequence
  SITE 23: note = Side chain of lysine linked via an amide linkage to another sequence
  SITE 23: note = Lysine carboxamide
KHKHHKHHKHHKHHKHHKHKKKK

SEQ ID NO: 66 - protein, 22 aa, synthetic construct
  SITE 20: note = Side chain of lysine linked via an amide linkage to another sequence
  SITE 21: note = Side chain of lysine linked via an amide linkage to another sequence
  SITE 22: note = Side chain of lysine linked via an amide linkage to another sequence
  SITE 22: note = Lysine carboxamide
KHKHKHKHKHKHKHKHKHKKKK

SEQ ID NO: 67 - protein, 32 aa, synthetic construct
  SITE 17: note = Side chain of lysine linked via an amide linkage to another sequence
  SITE 27: note = Side chain of lysine linked via an amide linkage to another sequence
  SITE 28: note = Side chain of lysine linked via an amide linkage to another sequence
  SITE 29: note = Side chain of lysine linked via an amide linkage to another sequence
  SITE 32: note = Aspartic acid carboxamide
HHHKHHHKHHHKHHHKKHHHHNHHHHKKKRGD

SEQ ID NO: 68 - protein, 33 aa, synthetic construct
  SITE 18: note = Side chain of lysine linked via an amide linkage to another sequence
  SITE 28: note = Side chain of lysine linked via an amide linkage to another sequence
  SITE 29: note = Side chain of lysine linked via an amide linkage to another sequence
  SITE 30: note = Side chain of lysine linked via an amide linkage to another sequence
  SITE 33: note = Aspartic acid carboxamide
KHHHKHHHKHHHKHHHKKHHHHNHHHHKKKRGD

SEQ ID NO: 69 - protein, 24 aa, synthetic construct
  SITE 17: note = Side chain of lysine linked via an amide linkage to another sequence
  SITE 19: note = Side chain of lysine linked via an amide linkage to another sequence
  SITE 20: note = Side chain of lysine linked via an amide linkage to another sequence
  SITE 21: note = Side chain of lysine linked via an amide linkage to another sequence
  SITE 24: note = Aspartic acid carboxamide
HHHKHHHKHHHKHHHKKGKKKRGD

SEQ ID NO: 70 - protein, 25 aa, synthetic construct
  SITE 16: note = Side chain of lysine linked via an amide linkage to another sequence
  REGION 1..25: note = This sequence is one part of a branched amino acid sequence
HHHKHHHKHHHKHHHKHHHHNHHHH

SEQ ID NO: 71 - protein, 26 aa, synthetic construct
  SITE 17: note = Side chain of lysine linked via an amide linkage to another sequence
  REGION 1..26: note = This sequence is one part of a branched amino acid sequence
HHHKHHHKHHHKHHHKKHHHHNHHHH

SEQ ID NO: 72 - protein, 27 aa, synthetic construct
  SITE 18: note = Side chain of lysine linked via an amide linkage to another sequence
  REGION 1..27: note = This sequence is one part of a branched amino acid sequence
KHHHKHHHKHHHKHHHKKHHHHNHHHH

SEQ ID NO: 73 - protein, 18 aa, synthetic construct
  SITE 17: note = Side chain of lysine linked via an amide linkage to another sequence
  REGION 1..18: note = This sequence is one part of a branched amino acid sequence
HHHKHHHKHHHKHHHKKG

SEQ ID NO: 74 - protein, 22 aa, synthetic construct
  SITE 13: note = Side chain of lysine linked via an amide linkage to another sequence
  REGION 1..22: note = This sequence is one part of a branched amino acid sequence
HHHKHHHKHHHKKHHHHNHHHH

SEQ ID NO: 75 - protein, 28 aa, synthetic construct
  SITE 13: note = Side chain of lysine linked via an amide linkage to another sequence
  SITE 23: note = Side chain of lysine linked via an amide linkage to another sequence
  SITE 24: note = Side chain of lysine linked via an amide linkage to another sequence
  SITE 25: note = Side chain of lysine linked via an amide linkage to another sequence
  SITE 28: note = Aspartic acid carboxamide
HHHKHHHKHHHKKHHHHNHHHHKKKRGD

(19) United States
(12) Patent Application Publication
(10) Pub. No.: US 2024/0158794 A1
(43) Pub. Date: May 16, 2024

(54) NUCLEOSIDE CONTAINING siRNAS FOR TREATING VIRAL DISEASES

(71) Applicant: Sirnaomics, Inc., Germantown, MD (US)

(72) Inventor: David M. EVANS, Germantown, MD (US)

(21) Appl. No.: 18/126,145

(22) Filed: Mar. 24, 2023

Related U.S. Application Data
(63) Continuation of application No. PCT/US21/53440, filed on Oct. 4, 2021.
(60) Provisional application No. 63/087,165, filed on Oct. 2, 2020.

(51) Int. Cl. C12N 15/113; A61K 31/7105

(52) U.S. Cl. CPC C12N 15/1131; A61K 31/7105; C12N 2310/14; C12N 2310/33

(57) ABSTRACT

Oligonucleotides, including siRNA duplex molecules are provided containing one or more antiviral nucleoside analogs. The analogs may be positioned within the oligonucleotide sequence or may be appended to one or more termini of the oligonucleotides. Pharmaceutical compositions containing the oligonucleotides are provided, together with methods of using the compositions for treating viral infections.

### CROSS REFERENCE TO RELATED APPLICATIONS

This application is a continuation of International Application No. PCT/US21/53440, filed on Oct. 4, 2021, which claims the benefit of U.S. Provisional Application No. 63/087,165, filed on Oct. 2, 2020, the entire contents of which are hereby incorporated by reference.

### SEQUENCE LISTING

The instant application contains a Sequence Listing which has been submitted electronically in XML format and is hereby incorporated by reference in its entirety. The XML file, created on Jul. 13, 2023, is named 4690_0031C_SL.xml and is 127,998 bytes in size.

### FIELD

Molecules, pharmaceutical compositions and methods of manufacture and use are provided for inhibiting the expression of genes of interest involved in viral infections.

### BACKGROUND

Antiviral nucleoside and nucleotide analogs have been developed against a variety of viral diseases. The analogs exert their antiviral effect by incorporation into nucleic strands and terminate synthesis of those strands.

siRNAs are double stranded RNA molecules consisting of a sense strand and a complementary antisense strand. These molecules may be blunt ended molecules that are 19-29 bases long on each strand or they may exhibit two base overhangs (typically dTdT). Each strand of the siRNA typically is made via solid-phase synthesis by conjugating consecutive bases in a desired sequence to the previous base attached to the growing oligonucleotide. Once synthesized, the two strands are annealed to each other to form the duplex. Amidite chemistry or other synthetic approaches are well known in the field and commercial services provide synthetic siRNA synthesis.

siRNAs against select targets within cancer cells, for example, have been shown to reduce expression of a protein encoded by the silenced gene target. Silencing these genes can in turn inhibit growth of that cell. If the cell is specifically a diseased cell (e.g., a cell infected with a virus) that the siRNA can access, then the siRNA may act as a therapeutic. Furthermore, in some cases, the use of select therapeutics (small molecule inhibitors, monoclonal antibodies, etc.) that are currently the ‘gold standard’ for therapy can be augmented by silencing genes in select pathways using siRNA methods.

It has previously been shown that the pyrimidine-based, non-native nucleoside analog, gemcitabine, (2′, 2′-difluoro 2′-deoxycytidine) (“GEM”), can replace certain bases in the sequence of an siRNA. GEM, when administered systemically, is taken up by nucleoside transporters, activated by tri-phosphorylation by deoxycytidine kinase and can then be incorporated into either RNA or DNA. It replaces the nucleic acid cytidine during DNA replication (cell division).

### SUMMARY OF THE CLAIMS

The disclosed embodiments are provided for molecules, pharmaceutical compositions and methods of manufacture and use (e.g., in treatment of subjects) in gene silencing. The compositions comprise gemcitabine and one or more nucleic acids such as siRNAs or miRNAs encapsulated in a histidine-lysine copolymer, the combination of which is formed into nanoparticles. Methods of use include treating subjects using the pharmaceutical composition for a variety of viral-related infections, including, e.g., HBV infection.

Specifically, what is provided is an oligonucleotide molecule containing nucleotides and one or more antiviral nucleoside analogs. The nucleotides may comprise deoxyribonucleotides and/or ribonucleotides. The oligonucleotide advantageously is an siRNA duplex.

The one or more antiviral nucleoside analogs may be attached to the 3′ or 5′ end of the molecule, or may be present within the molecule. The nucleoside analog may be selected from the group consisting of Abacavir, Acyclovir, Adefovir, Cidofovir, Clevudine, cytarabine, Didanosine, didanosine (ddI), emtricitabine, Emtricitabine, Entecavir, Famciclovir, galidesivir, Ganciclovir/Valganciclovir, gemcitabine, GS-441524, idoxuridine, lamivudine (3TC), Molnupiravir, Remdesivir, Ribavirin, Sofosbuvir, stavudine (d4T), Telbivudine, Tenofovir, trifluridine, Valacyclovir, vidarabine, zalcitabine (ddC), and Zidovudine.

Also provided are pharmaceutical compositions comprising an oligonucleotide as described above, optionally containing a histidine-lysine copolymer.

Further provided are methods of treating a viral infection in a subject, by administering to the subject an effective amount of a pharmaceutical composition as described above. The subject may be a mammal, such as a human.

### BRIEF DESCRIPTION OF THE FIGURES

The disclosed embodiments are more readily understood with reference to the embodiments illustrated in the following figures.

FIGS. 1A-1G show examples of histidine-lysine copolymers that may be used in some of the embodiments disclosed. These figures disclose H3K4b (FIG. 1(a)) (“KHHHKHHHKHHHKHHHK” disclosed as SEQ ID NO: 25, and “KHHHKHHHKHHHKHHHKKKK” disclosed as SEQ ID NO: 64), H2K4b (FIG. 1(b)) (“KHKHHKHHKHHKHHKHHKHK” disclosed as SEQ ID NO: 24 and “KHKHHKHHKHHKHHKHHKHKKKK” disclosed as SEQ ID NO: 65), HK4b (FIG. 1(c)) (“KHKHKHKHKHKHKHKHKHK” disclosed as SEQ ID NO: 55 and “KHKHKHKHKHKHKHKHKHKKKK” disclosed as SEQ ID NO: 66), H3K8b (FIG. 1(d)); (“HHHHNHHHH” disclosed as SEQ ID NO: 43, “HHHKHHHKHHHKHHH” disclosed as SEQ ID NO: 51, “HHHKHHHKHHHKHHHKHHHHNHHHH” disclosed as SEQ ID NO: 70, and “HHHKHHHKHHHKHHHKHHHHNHHHHKKK” disclosed as SEQ ID NO: 62), H3K8b(+RGD) or (K+)H3K8b(+RGD) (FIG. 1(e)) (“HHHKHHHKHHHKHHHK” disclosed as SEQ ID NO: 56, “KHHHKHHHKHHHKHHHK” disclosed as SEQ ID NO: 25, “HHHKHHHKHHHKHHHKKHHHHNHHHH” disclosed as SEQ ID NO: 71, “KHHHKHHHKHHHKHHHKKHHHHNHHHH” disclosed as SEQ ID NO: 72, “HHHKHHHKHHHKHHHKKHHHHNHHHHKKKRGD” disclosed as SEQ ID NO: 67, and “KHHHKHHHKHHHKHHHKKHHHHNHHHHKKKRGD” disclosed as SEQ ID NO: 68), H3K(G)8b (FIG. 1(f)) (“HHHKHHHKHHHKHHHK” disclosed as SEQ ID NO: 56, “HHHKHHHKHHHKHHHKKG” disclosed as SEQ ID NO: 73, and “HHHKHHHKHHHKHHHKKGKKKRGD” disclosed as SEQ ID NO: 69) and (−HHHK)H3K8b or (−HHHK)H3K8b(+RGD) (FIG. 1(g))(“HHHKHHHKHHHK” disclosed as SEQ ID NO: 57, “HHHKHHHKHHHKKHHHHNHHHH” disclosed as SEQ ID NO: 74, “HHHKHHHKHHHKKHHHHNHHHHKKK” disclosed as SEQ ID NO: 63, and “HHHKHHHKHHHKKHHHHNHHHHKKKRGD” disclosed as SEQ ID NO: 75), in order of appearance.

FIG. 2 shows examples of nucleoside analogs that may be used in some of the embodiments disclosed (from: https://pubs.acs.org/doi/10.1021/cr5002035).

FIG. 3 shows examples of prodrug nucleosides that may be used in some of the embodiments disclosed.

FIG. 4 shows examples carbonyloxymethyl nucleotide prodrugs approved by the FDA or in clinical trials that may be used in some of the embodiments disclosed.

FIG. 5 shows the synthesis of the HepDirect Prodrug of Lamivudine. Compounds S-223 and 227 form compound 228 under conditions include Et₃N THF at −40 degrees C. to room temperature (48%). Compound 228 then undergoes a two-step reaction to form a mixture of (two) enantiomers shown as compound 229. The first step requires 3TC 5-(methylthio)-1H-tetrazole DMF for 30 minutes at room temperature. The second step requires 5-6 M t-BuOOH in heptane between −40 degrees to 25 degrees C. for three hours (70%).

### DETAILED DESCRIPTION

It has been found that antiviral nucleoside analogs can replace certain nucleotides in the sequence of siRNAs targeting viral-related mRNAs, e.g. hepatitis B. Silencing of viral gene expression augments activity of the nucleoside released from the siRNA.

The analogs may be added to, for example, the Sense strand (“SS”) of the siRNA when the Antisense strand (“AS”) is released and bound to the RNA-induced silencing complex (“RISC complex”) to induce gene silencing. Antiviral nucleoside analogs may also be added into the AS strand without impacting activity of the AS strand in silencing the gene. A variety of nucleoside analogs may substitute for naturally occurring nucleosides within an siRNA sequence. Alternatively, the analogs may can be added to the 3′ or 5′ end s of the SS and AS strands where they are released when the siRNA is processed in the cytoplasm of the cells. Combining analogs on both the SS and AS shows even greater efficacy.

In some embodiments methods are provided for making pharmaceutical compositions comprising HKP, HKP(+H) or any other histidine-lysine copolymer and a siRNA solution, which, when mixed, spontaneously form nanoparticles. Viral diseases can be modulated by siRNA silencing of specific gene targets present in the virus. For example, an siRNA against HBV may also include the nucleoside analog lamivudine as a part of the structure of the sense strand (or AS strand) of the siRNA targeting the HBV gene. Consequently, when the siRNA is administered to hepatocyte cells in the liver where HBV resides the Sense strand is released from the double stranded siRNA and is degraded by nucleases present within the cytoplasm of the cells. The AS strand is engaged in RISC and surveils for mRNA (or viral RNA) sequences and produces silencing by cleavage of the sequence having identity with the AS strand.

Molecules, compositions and methods are provided for silencing genes in diseases and disorders stemming from viral infections. The compositions contain one or more nucleic acids such as siRNA or miRNA where the nucleic acid is modified by the presence of one or more nucleoside analogs, together with a histidine-lysine copolymer. Nanoparticles are formed when the components of the compositions are mixed using a microfluidic mixer. The methods may be used to block the expression of a variety of viral genes, inhibiting viral replication and providing methods of ameliorating or eradicating the viral infection.

### Definitions

Small interfering RNA (siRNA): a duplex oligonucleotide that is a short, double-stranded RNA that interferes with the expression of a gene in a cell, after the molecule is introduced into the cell. For example, it targets and binds to a complementary nucleotide sequence in a single stranded target RNA molecule. SiRNA molecules are chemically synthesized or otherwise constructed by techniques known to those skilled in the art. Such techniques are described in U.S. Pat. Nos. 5,898,031, 6,107,094, 6,506,559, 7,056,704, RE46,873 E, and 9,642,873 B2 and in European Pat. Nos. 1214945 and 1230375, all of which are incorporated herein by reference in their entireties. By convention in the field, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

MicroRNA (miRNA): a small, non-coding RNA molecule that functions in RNA silencing and post-transcriptional regulation of gene expression by targeting and binding to a complementary nucleotide sequence in a single-stranded target RNA molecule.

Anti-sense oligonucleotide (ASO): a short, single-stranded RNA or DNA (typically 11-27 bases) that can reduce expression of a gene within a mammalian cell by targeting and binding to a complementary nucleotide sequence in a single-stranded target RNA molecule.

A DNA or RNA aptamer: a single-stranded DNA or RNA oligonucleotide that binds to a specific target molecule. Such targets include small molecules, proteins, and nucleic acids. Such aptamers are usually created from a large random sequence pool through repeated rounds of in vitro selection or systematic evolution of ligands by exponential enrichment (SELEX).

Histidine-lysine copolymer: a peptide or polypeptide consisting of histidine and lysine amino acids. Such copolymers are described in U.S. Pat. Nos. 7,070,807 B2, 7,163,695 B2, and 7,772,201 B2, which are incorporated herein by reference in their entireties.

RISC complex is the RNA-induced silencing complex, a multi-component structure that is active in a number of pathways involved in gene silencing (both transcriptional and translational). The siRNA serves as a template for the RISC to recognize the complementary RNA strand and target it for degradation.

Nucleosides and Modifications

Recently, GalNAc modified siRNAs have been used to promote delivery of these siRNAs specifically to hepatocytes within the liver. The GalNac moieties bind with very high affinity to the asialoglycoprotein receptors (ASGPR) present specifically and at high numbers on the hepatocytes. The ASGPRs are believed to be internalized into the cells upon binding and therefore carry the attached siRNA into the cell with them.

Other targeting ligands that can deliver a payload to specific cell types include the GLP1 peptide (binding to the GLP1 receptor on Pancreatic Beta cells), RGD motifs (e.g. cRGD, iRGD that bind α5β3 integrin receptors or peptides derived from the Foot and Mouth virus (binding with nM affinity to α5β6 integrin receptors compared with almost micromolar affinity for α5β3 receptors)), Folate ligands (that bind to folate receptors), Transferrin ligands binding to Transferrin receptors and EGFR targeting through EGF receptors. Many other examples of targeting moieties show specificity for delivery to distinct cell types.

Compositions and methods are described herein that provide co-delivery of an siRNA (that will silence a gene) along with an antiviral nucleoside analog drug (e.g. lamivudine) to produce a therapeutic benefit that is greater than administration of either the siRNA or the drug alone.

Gemcitabine (like 5-FU and other nucleoside analogs) can be chemically synthesized in a manner to allow direct coupling to DNA or RNA bases through traditional synthetic means (manually or by using automated instruments).

Incorporation of potent nucleoside analogs into the SS or AS strand of the siRNA allows for a double effect of the therapeutic-release of the nucleoside analogs from the siRNA induces inhibition of the HBV, while silencing of the RNA from the virus further potentiates the effect of the drug in reducing viral load within the cells.

Examples of non-native nucleoside analogs that can be incorporated into siRNA sequences for use as antiviral agents include:
Clevudine (a thymidine analog that can be used to replace uracil moieties within the sequence and that can still base-pair with the corresponding base (A) in the alternative strand);
Entecavir, a carboxylic analogue of guanosine that can replace G within the siRNA sequence while still allowing base-pairing with the base “C,”
Lamivudine, a non-native nucleoside with activity against HBV, is an analogue of “C” that will base-pair with “G”s on the alternative strand.

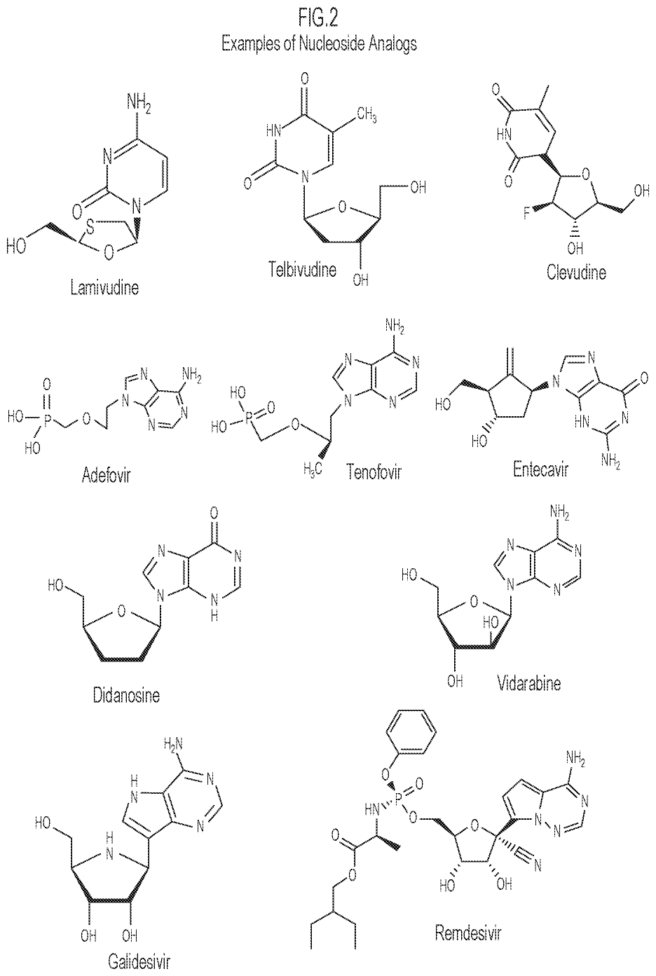

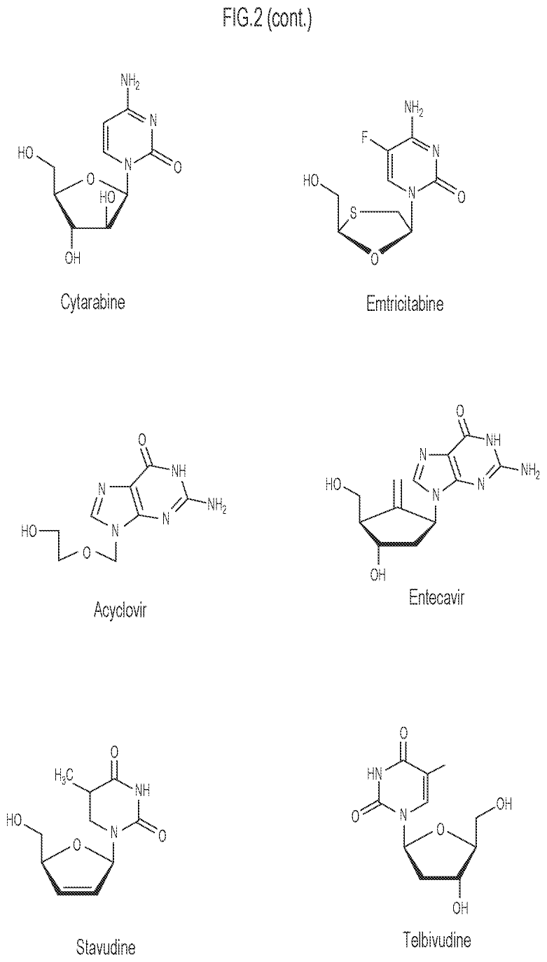

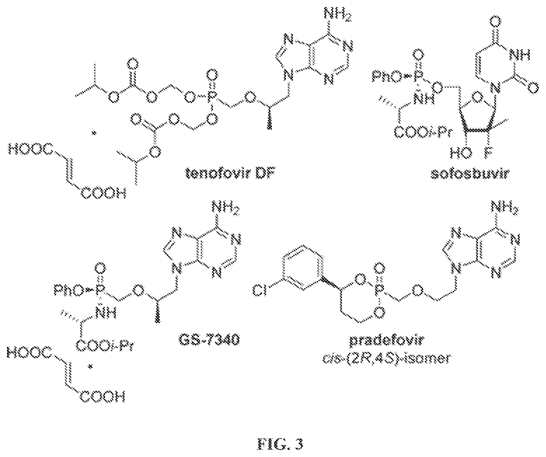

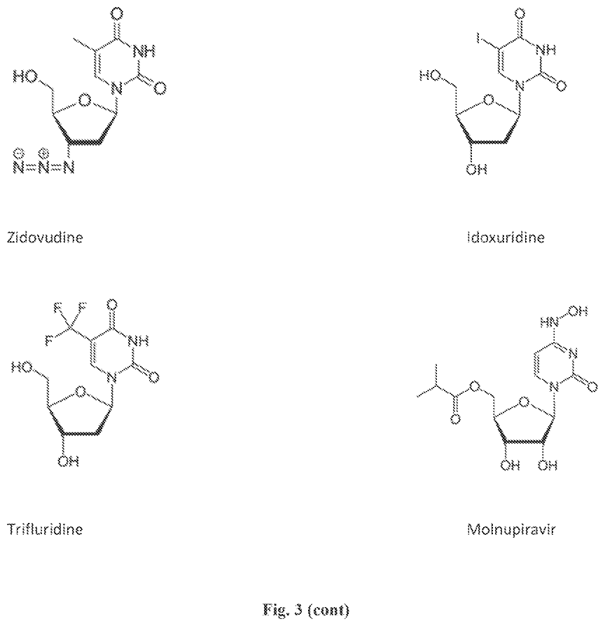

Other analogs that may be used in the embodiments described herein include: Abacavir (viral target HIV); Acyclovir (HSV, VZV); Adefovir (HBV); Cidofovir (CMV); Didanosine (HIV); Emtricitabine (HIV, HBV); Famciclovir (HSV, VZV); Ganciclovir/Valganciclovir (CMV), Remdesivir (COVID); Sofosbuvir (HCV); Stavudine (REV); Telbivudine (HBV); Tenofovir (HIV, HBV); Valacyclovir (HSV, VZV), Zidovudine (HIV); Ribavirin (RSV, HCV); GS-441524 (related to remdesivir); and Molnupiravir (COVID). The structures of these molecules are well known and are shown in FIGS. 2 and 3.

These nucleosides analog may be inserted within an siRNA sequence to replace the naturally occurring residue in the same manner as described above for clevudine, entecavir and lamivudine. The corresponding naturally occurring residue for any given nucleoside analog is well known in the art. Examples include:
deoxyadenosine analogues:
   didanosine (ddI)
   vidarabine
adenosine analogues:
   galidesivir
   remdesivir
deoxycytidine analogues:
   cytarabine
   gemcitabine
   emtricitabine
   lamivudine (3TC)
   zalcitabine (ddC)
guanosine and deoxyguanosine analogues:
   abacavir
   aciclovir
   entecavir
thymidine and deoxythymidine analogues:
   stavudine (d4T)
   telbivudine
   zidovudine (azidothymidine, or AZT)
deoxyuridine analogues:
   idoxuridine
   trifluridine

These nucleosides may be situated within the sequence of an siRNA in place of the natural bases as described above, or may be appended to the 3′ or 5′ end of each strand. Single of multiple copies of the analog can be appended to the 3′ or 5′ end of either the SS or the AS strand of siRNAs which results in specific release of these molecules when internalized within the cells. Advantageously 1, 2, 3, or 4 analogs may be appended to the 3′ and/or 5′ end of the AS or SS.

Some analogs that lack one of the functional groups that correspond to the 3′ and 5′ hydroxyl groups of RNA nucleosides cannot be inserted within siRNA sequences because they cannot form two phosphodiester linkages. For example, emtricitabine and acyclovir both only have hydroxyl groups equivalents to the 5′ hydroxyl. Accordingly, such residues are appended at the termini of siRNA molecules—for example emtricitabine and acyclovir would be linked via their 5′ hydroxyl groups at the 3′ ends of either the AS or SS, or both.

The analogs may be coupled to an siRNA chain using standard chemical methods are well known in the art. For example, standard phosphoramidite coupling chemistry is well known in the art and is readily applied to the analogs described herein.

The bases of the siRNA containing the modified nucleosides can be unmodified or chemically modified to improve stability against nucleases. For example, nucleosides that are modified at the 2′01-1 group with either 2′OMe or 2′Fluoro modifications (or other modifications that are resistant to enzyme degradation of the strands) may be used since these modification confer nuclease resistance on the siRNA.

Furthermore, incorporation of phosphorothioates can improve the resistance to nucleases. Monovalent phosphorothioates can be used, but produce diastereomeric mixtures that are undesirable. Consequently PS2 (dithio phosphorothioates) can be utilized to improve stability without introduction of stereochemical alterations in the molecule.

Some of the examples described below are aimed at HBV, however it is also possible to vary the siRNA sequences to target additional viruses while incorporating non-native nucleoside analogs into these structures that inhibit these viruses. For example, lamivudine is used to treat HIV as well as HBV and it is possible to combine this non-native nucleoside with an siRNA targeting the HIV genome. Alternatively, the siRNAs may be targeted against human host factors that allow replication of the viruses or otherwise enable viral infection to proceed in humans or animal hosts. It is possible to incorporate one or more than one non-native nucleoside analog into each siRNA sequence.

Delivery of the siRNA structures can be achieved, for example, via GalNAc conjugation directly to the siRNA containing the non-native nucleosides. It may also be achieved via other constructs such as: using the Peptide Docking Vehicle coupled to GalNAc; via administration in a lipid nanoparticle; via use of Histidine Lysine branched polymer nanoparticles; or by other ligands immobilized directly to the chemically stabilized siRNA sequence.

The structures of lamivudine, clevudine and entecavir are shown in FIG. 2 and these molecules are further described in J. Antimicrob. Chemother. 66:2715-2725 (2011).

Clevudine

Clevudine [2′-fluoro-5-methyl-b-L-arabinofuranosyl-uracil (L-FMAU)] is a thymidine analogue, and therefore is structurally similar to telbivudine. Clevudine has a fluoride group at the 2′ position on the furanose moiety in place of hydrogen which is found in telbivudine. It undergoes stepwise phosphorylation to its active triphosphate metabolite. Based on two Phase III trials with only 6 months of treatment, clevudine was approved in South Korea in 2006 and was approved in the Philippines in 2009. Its proposed mechanism of action includes targeting HBV DNA polymerase, reverse transcriptase and the conversion of partially double-stranded DNA into covalently closed circular DNA (cccDNA). This reduction in cccDNA combined with its long half-life may contribute to the post-treatment effect of clevudine where viral suppression is maintained for a period of time even after stopping therapy. Development of clevudine has been halted due to occurrences of myopathy and mitochondrial toxicity occurring after several months of treatment.

Entecavir

Entecavir is approved for treatment of treatment-naive and lamivudine resistant CHB. Entecavir is a carboxylic analogue of guanosine that undergoes intracellular phosphorylation to its active 5′ triphosphate metabolite. This competes with the natural substrate deoxyguanosine triphosphate and inhibits HBV DNA polymerase. In vitro studies have shown that entecavir inhibits HBV DNA polymerase priming, which involves guanosine (an additional antiviral mechanism compared with other NAs), reverse transcription of the pre-genomic messenger RNA and synthesis of the positive-stranded HBV DNA. In contrast to other NAs that are obligate chain terminators, the active moiety in entecavir contains a 3′-hydroxyl group that allows a few additional nucleotides to be incorporated prior to chain termination. Therefore entecavir is a non-obligate chain terminator. Entecavir is more effective than lamivudine in reducing viral DNA replication in vitro and has shown higher rates of virological suppression in subjects. A trial of 715 treatment-naive HBeAg-positive patients showed a significantly higher rate of histological, virological and biochemical improvement in those treated with entecavir compared with lamivudine. Similar results were obtained in the Phase III study with 648 treatment-naive HBeAg-negative CHB patients.

Lamivudine

Lamivudine [2′,3′-dideoxy-3′thiacytidine (3TC)] is approved for the treatment of chronic hepatitis B (CHB) and was the first oral nucleoside analog available for CHB. It is an analogue of cytidine, and is phosphorylated to its active metabolites, acting as a chain terminator after competing for incorporation into viral DNA. Lamivudine is effective in the normalization of ALT, HBeAg seroconversion, suppressing HBV DNA and reversing fibrosis.

Common side effects include nausea, diarrhea, headaches, fatigue and cough. Serious side effects include liver disease, lactic acidosis, and worsening hepatitis B among those already infected. It is safe for people over three months of age and can be used during pregnancy. The medication can be taken with or without food. Lamivudine is a nucleoside reverse transcriptase inhibitor and works by blocking the HIV reverse transcriptase and hepatitis B virus polymerase.

Lamivudine is an analogue of cytidine. It can inhibit both types (1 and 2) of HIV reverse transcriptase and also the reverse transcriptase of hepatitis B virus. It is phosphorylated to active metabolites that compete for incorporation into viral DNA. They inhibit the HIV reverse transcriptase enzyme competitively and act as a chain terminator of DNA synthesis. The lack of a 3′—OH group in the incorporated nucleoside analogue prevents the formation of the 5′ to 3′ phosphodiester linkage essential for DNA chain elongation, and therefore, the viral DNA growth is terminated.

Synthesis

Method of synthesizing amidites of nucleoside analogs have been described (e.g. https://www.ncbi.nlm.nih.gov/pmc/articles/PMC6273010/) and these amidites can be used to incorporate nucleoside analogs into siRNA sequences.

Some examples of prodrug nucleosides are shown in FIG. 3 (from: https://pubs.acs.org/doi/10.1021/cr5002035).

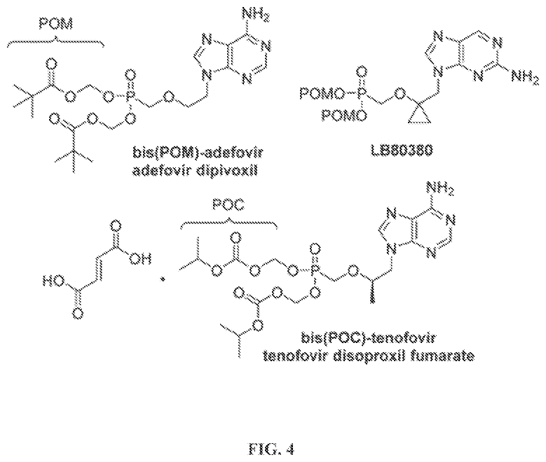

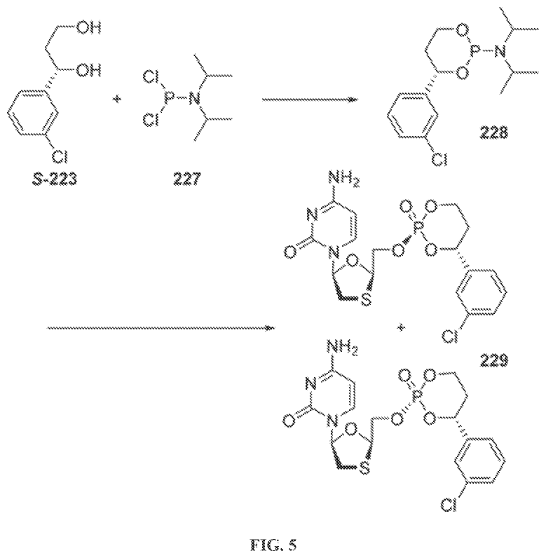

Additional examples of carbonyloxymethyl nucleotide prodrugs approved by the FDA or in clinical trials are shown in FIG. 4.

Synthesis of the HepDirect Prodrug of Lamivudine is shown in FIG. 5. Phosphoramidite 228 is synthesized by reaction of diol S-223 and commercially available 1,1-dichloro-N,N-diisopropylphosphinamine 227. The desired HepDirect prodrug of Lamivudine 229 is obtained as a mixture of cis- and trans-phosphate cyclic diesters after coupling of phosphoramidite 228 with 3TC followed by oxidation with t-BuOOH.

Any of these structures can be incorporated into the 3′ or 5′ end of the SS or AS strand of an siRNA without the need to hybridize with a cognate base on the opposing strand. Codelivery with the siRNA will augment activity between both agents on producing an antiviral response. The skilled artisan will recognize that other chemical agents could be introduced into the siRNA structure that would augment the activity against other viruses.

Examples are shown below of antiviral siRNA molecules that can be modified by the introduction of antiviral nucleoside analogs as described above.

COVID-19:

| | SEQ | siRNA Sequence | SEQ |
|---|---|---|---|
| siRNA Sequence | ID | (anti-sense): | ID |
| (sense): (5′-3′) | NO | (5′-3′) | NO. |
| UCAGUGUGUUAA | 1 | UGUAAGAUUAA | 6 |
| UCUUACAdTdT | | CACACUGAdT | |
| | | dT | |
| UAAUCUUACAAC | 2 | AGUUCUGGUUG | 7 |
| CAGAACUdTdT | | UAAGAUUAdT | |
| | | dT | |
| UUUCACACGUGG | 3 | AUAAACACCAC | 8 |
| UGUUUAUdTdT | | GUGUGAAAdT | |
| | | dT | |
| UGUUACUUGGUU | 4 | AGCAUGGAACC | 9 |
| CCAUGCUdTdT | | AAGUAACAdT | |
| | | dT | |
| CAAUGGUACUAA | 5 | AAACCUCUUAG | 10 |
| GAGGUUUdTdT | | UACCAUUGdT | |
| | | dT | |

| HBV: | SEQ ID NO. |
|---|---|
| GAGGACUCUUGG | 11 |
| ACUCUCA | |
| UGUCAACGUCCG | 12 |
| ACCUUGA | |
| CGUCCGACCUUG | 13 |
| AGGCAUA | |
| UGAUCUUUGUAC | 14 |
| UAGGAGG | |
| AUUGGUCUGUUC | 15 |
| ACCAGCA | |
| HIV (3′ to 5′): | SEQ ID NO. |
| GAACAUGGACUU | 16 |
| GUAUAUU | |
| GGACGAGAAAGA | 17 |
| UCAUUGA | |
| GGUAGGAUCAGC | 18 |
| CCUCAUU | |
| CCCGAGGGCGAG | 19 |
| GAUGAGAAA | |
| GGAAACUGCUGC | 20 |
| UGUGUAC | |
| GGUACGACUCUG | 21 |
| GCAUUGAG | |
| GAAUUGCUGCUU | 22 |
| CGGAAUG | |
| GAAGGGAAGUUU | 23 |
| CAGUAA | |

### Histidine-Lysine (HK) Copolymers

Effective means for transferring nucleic acids into target cells are important tools, both in the basic research setting and in clinical applications. A diverse array of nucleic acid carriers is currently required because the effectiveness of a particular carrier depends on the characteristics of the nucleic acid that is being transfected [Blakney et al. Biomacromolecules 2018, 19: 2870-2879. Goncalves et al. Mol Pharm 2016; 13: 3153-3163. Kauffman et al. Biomacromolecules 2018; 19: 3861-3873. Peng et al. Biomacromolecules 2019; 20: 3613-3626. Scholz et al. J Control Release 2012; 161: 554-565]. Among various carriers, non-viral delivery systems have been developed and reported to be more advantageous than the viral delivery system in many aspects [Brito et al. Adv Genet. 2015; 89: 179-233]. For example, the large molecular weight branched polyethylenimine (PEI, 25 kDa) is an excellent carrier for plasmid DNA but not for mRNA. However, by decreasing the molecular weight of PEI to 2 kDa, it becomes a more effective carrier of mRNA [Bettinger et al. Nucleic Acids Res 2001; 29: 3882-3891].

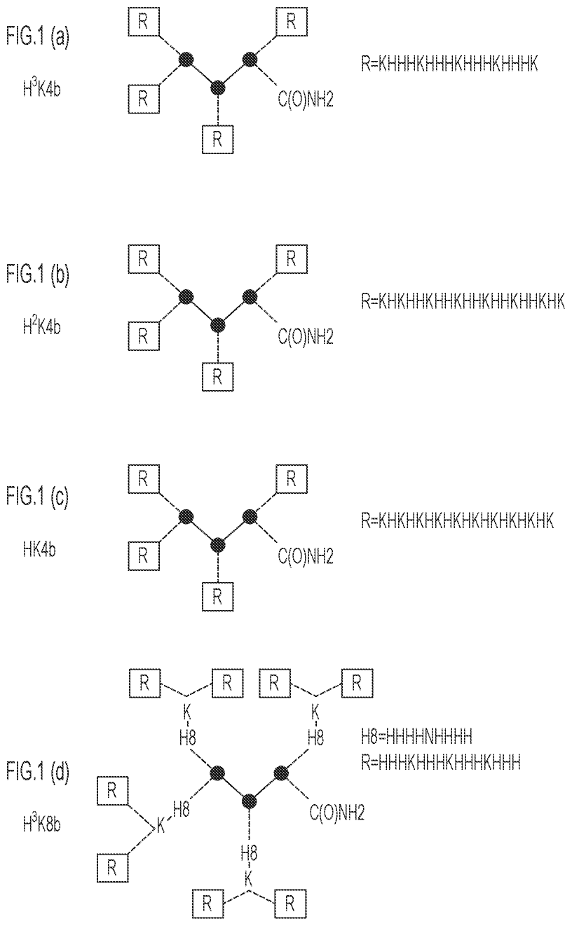

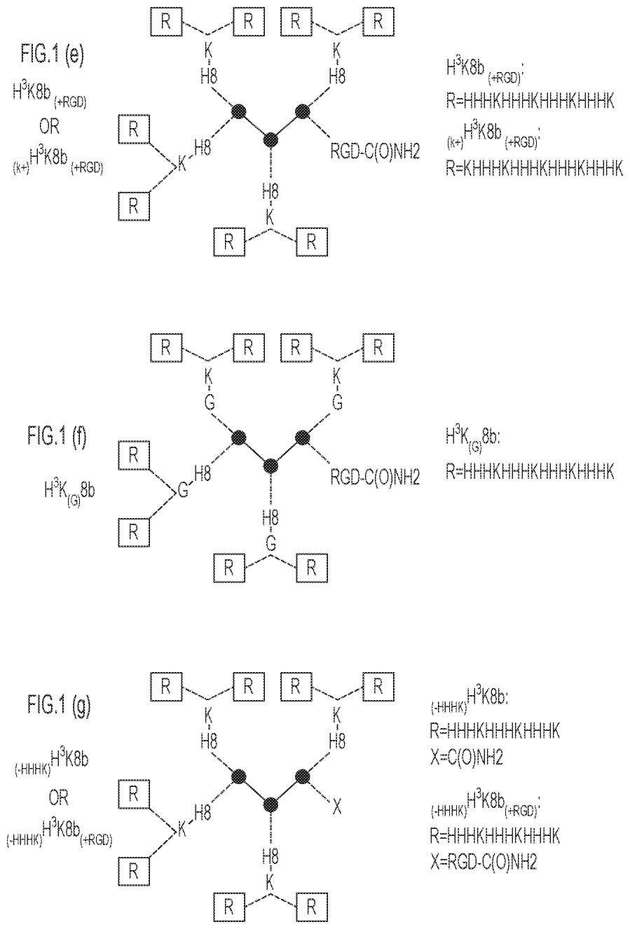

The four-branched histidine-lysine (HK) peptide polymer H²K4b has been shown to be a good carrier of large molecular weight DNA plasmids [Leng et al. Nucleic Acids Res 2005; 33: e40.], but a poor carrier of relatively low molecular weight siRNA [Leng et al. J Gene Med 2005; 7: 977-986.]. Two histidine-rich peptides analogs of H²K4b, namely H³K4b and H³K(+H)4b, were shown to be effective carriers of siRNA [Leng et al. J Gene Med 2005; 7: 977-986. Chou et al. Biomaterials 2014; 35: 846-855.], although H³K(+H)4b appeared to be modestly more effective [Leng et al. Mol Ther 2012; 20: 2282-2290]. Moreover, the H³K(+H)4b carrier of siRNA induced cytokines to a significantly lesser degree in vitro and in vivo than H³K4b siRNA polyplexes [Leng et al. Mol Ther 2012; 20: 2282-2290], which were already at very low levels. Suitable HK polypeptides are described in WO/2001/047496, WO/2003/090719, and WO/2006/060182, the contents of each of which are incorporated herein in their entireties. These polypeptides have a lysine backbone (three lysine residues) where the lysine side chain C-amino groups and the N-terminus are coupled to various HK sequences. HK polypeptide carriers can be synthesized by methods that are well-known in the art including, for example, solid-phase peptide synthesis (SPPS). FIG. 1 shows examples of several HK polymer structures that can be used in the disclosed composition and method embodiments.

It was found that such histidine-lysine peptide polymers (“HK polymers”), in addition to their ability to package and carry siRNAs, also are effective as mRNA carriers, and they can be used, alone or in combination with liposomes, to provide effective delivery of mRNA into target cells. Similar to PEI and other carriers, initial results suggested HK polymers differ in their ability to carry and release nucleic acids. However, because HK polymers can be reproducibly made on a peptide synthesizer, their amino acid sequence can be easily varied, thereby allowing fine control of the binding and release of siRNA, miRNA or mRNAs, as well as the stability of polyplexes containing the HK polymers and mRNA [Chou et al. Biomaterials 2014; 35: 846-855. Midoux et al. Bioconjug Chem 1999; 10: 406-411. Henig et al. Journal of American Chemical Society 1999; 121: 5123-5126.]. When siRNA, miRNA, or mRNA molecules are admixed with one or more HKP carriers the components self-assemble into nanoparticles.

As described for certain embodiments, one example of an HK polymer comprises four short peptide branches linked to a three-lysine amino acid core. The peptide branches consist of histidine and lysine amino acids, in different configurations. The general structure of these histidine-lysine peptide polymers (HK polymers) is shown in Formula I, where R represents the peptide branches and K is the amino acid L-lysine.

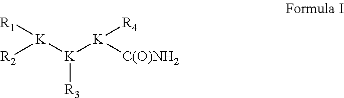

In Formula I where K is L-lysine and each of R₁, R₂, R₃ and R₄ is independently a histidine-lysine peptide. The R₁₋₄ branches may be the same or different in the HK polymers of the disclosed embodiments. When a R branch is “different”, the amino acid sequence of that branch differs from each of the other R branches in the polymer. Suitable R branches used in the HK polymers of the disclosed embodiments shown in Formula I include, but are not limited to, the following R branches R_{A}-R_{-J}:

(SEQ ID NO: 24)
R_{A} = KHKHHKHHKHHKHHKHHKHK-
(SEQ ID NO: 25)
R_{B} = KHHHKHHHKHHHKHHHK-
(SEQ ID NO: 26)
R_{c} = KHHHKHHHKHHHHKHHHK-
(SEQ ID NO: 27)
R_{D} = kHHHHHHHHHHHHHk-
(SEQ ID NO: 28)
R_{E} = HKHHHKHHHKHHHHKHHHK-
(SEQ ID NO: 29)
R_{F} = HHKHHHKHHHKHHHHKHHHK-
(SEQ ID NO: 30)
R_{G} = KHHHHKHHHHKHHHHKHHHHK-
(SEQ ID NO: 31)
R_{H} = KHHHKHHHKHHHKHHHHK-
(SEQ ID NO: 32)
R_{I} = KHHHKHHHHKHHHKHHHK-
(SEQ ID NO: 33)
R_{J} = KHHHKHHHHKHHHKHHHHK-

Specific HK polymers that may be used in the siRNA, miRNA and/or mRNA compositions include, but are not limited to, HK polymers where each of R1, R2, R3 and R4 is the same and selected from R_{A}-R_{J} (Table 1). These HK polymers are termed H²K4b, H³K4b, H³K(+H)4b, H³k(+H)4b, H-H³K(+H)4b, HH-H³K(+H)4b, H⁴K4b, H³K(1+H)4b, H³K(3+H)4b and H³K(1,3+H)4b, respectively. In each of these 10 examples, upper case “K” represents a L-lysine, and lower case “k” represents D-lysine. Extra histidine residues, in comparison to H³K4b, are underlined within the branch sequences. Nomenclature of the HK polymers is as follows:
1) for H³K4b, the dominant repeating sequence in the branches is -HHHK- (SEQ ID NO: 45), thus “H³K” is part of the name; the “4b” refers to the number of branches;
2) there are four -HHHK- motifs (SEQ ID NO: 45) in each branch of H³K4b and analogues; the first -HHHK- motif (SEQ ID NO: 45) (“1”) is closest to the lysine core;
3) H³K(+H)4b is an analogue of H³K4b in which one extra histidine is inserted in the second -HHHK- motif (SEQ ID NO: 45) (motif 2) of H³K4b;
4) for H³K(1+H)4b and H³K(3+H)4b peptides, there is an extra histidine in the first (motif 1) and third (motif 3) motifs, respectively;
5) for H³K(1,3+H)4b, there are two extra histidine residues in both the first and the third motifs of the branches.

TABLE 1
Examples of branched polymers

| | | Sequence |
|---|---|---|
| | | Identi- |
| Polymer | Branch Sequence | fier |
| H²K 4b | R_{A} = KHKHHKHHKHHKHHKHHKHK- | 34 |
| | 4 3 2 1 | |
| H³K4b | R_{B} = KHHHKHHHKHHHKHHHK- | 61 |
| H³K(+H)4b | R_{C} = KHHHKHHHKHHHHKHHHK- | 35 |
| H³k(+H)4b | R_{D} = kHHHkHHHkHHHHKHHHk- | 36 |
| H-H³K(+H)4b | R_{E} = HKHHHKHHHKHHHHKHHHK- | 37 |
| HH-H³K(+H)4b | R_{F} = HHKHHHKHHHKHHHHKHHHK- | 38 |
| H⁴K 4b | R_{G} = KHHHHKHHHHKHHHHKHHHHK- | 39 |
| H³K(1+H)4b | R_{H} = KHHHKHHHKHHHKHHHHK- | 40 |
| H³K(3+H)4b | R_{I} = KHHHKHHHHKHHHKHHHK- | 41 |
| H³K(1,3+H)4b | R_{J} = KHHHKHHHHKHHHKHHHHK- | 42 |

TABLE 2
Additional examples of HK Polymers

| Peptide Sequence | SEQ ID No. |
|---|---|
| HHHHNHHHH | 43 |
| HHHKHHHKHHHKHHHKHHH | 44 |
| HHHK | 45 |
| HHKHH | 46 |
| KHHHKHHHKHHHKHHHHHHHHHKHHHKHHHKHHHHNHHHHH | 47 |
| KHHHKHHHKHHHKHHHHHHKHHHKHHHKHHHKHHHHNHHHHHRGD | 48 |
| HHHKHHHKHHHHHHKHHHKHHHKHHHHNHHHHH | 49 |
| KHHHKHHHKHHHHHHKHHHKHHHKHHHHNHHHHH | 50 |
| HHHKHHHKHHHKHHH | 51 |
| HHHKHHHKHHH | 52 |
| KHHHKHHHKHHHKHHHK | 53 |
| KHKHHKHHKHHKHHKHHKHK | 54 |
| KHKHKHKHKHKHKHKHKHK | 55 |
| HHHKHHHKHHHKHHHK | 56 |
| HHHKHHHKHHHK | 57 |
| H³K8b | 51, 70, and |
| | 62 |
| (-HHHK)H³K8b | 57, 74, and |
| | 63 |

Methods well known in the art, including gel retardation assays, heparin displacement assays and flow cytometry can be performed to assess performance of different formulations containing HK polymer plus liposome in successfully delivering mRNA. Suitable methods are described in, for example, Gujrati et al., Mol. Pharmaceutics 11:2734-2744 (2014), Pärnaste et al., Mol Ther Nucleic Acids. 7: 1-10 (2017).

Detection of nucleic acid uptake into cells can also be achieved using SmartFlare® technology (Millipore Sigma). These smart flares are beads that have a sequence attached that, when recognizing the RNA sequence in the cell, produce an increase in fluorescence that can be analyzed with a fluorescent microscope. siRNAs can reduce expression of a target gene while mRNA can increase it. miRNAs can either increase or decrease expression.

Other methods include measuring protein expressions from the nucleic acid, for example, an mRNA encoding luciferase can be used to measure the efficiency of transfection using methods that are well known in the art. See, for example, this was accomplished with luciferase mRNA in a recent publication (He et al, J Gene Med. 2021 February; 23(2):e3295) to demonstrate the efficacy of delivering mRNA using a HKP and liposome formulation.

Excessive histidine-lysine copolymer in the pharmaceutical composition can have a toxic effect on subjects. A lower copolymer to siRNA ratio was selected to mitigate any toxicity that may result from administration.

A number of patterns of HK polymers that might be effective for siRNA, miRNA or mRNA transport were isolated, developed and evaluated. Among the polymers with 4 branches, the repeating pattern of HHHK (SEQ ID NO: 45) (e.g., H³K4b on the terminal branch appears to augment uptake of siRNA more effectively than the repeating patterns of HHK (e.g., H²K4b) or HK (e.g., HK4b). As a result, a similar pattern was adopted in constructing the highly branched HK8b and H³K8b and found it to be highly effective for preparing carriers of siRNA.

H³K8b has eight terminal branches, and has a high percentage of histidine residues and a low percentage of lysine residues. Compared to HHK, the pattern HHHK (SEQ ID NO: 45) has an increased buffering capacity because of the higher ratio of histidine residues, and reduced binding because of the lower ratio of lysine residues. An increased number of histidine residues in the terminal branches that buffer the acidic endosomal compartment would allow endosomal lysis and escape of DNA from the endosomes. Similarly, the histidine rich domain in H³K8b would be expected to increase cytosol delivery by enhancing the buffering capacity of the polymer. Nevertheless, replacement of the histidine-rich domain with a glycine or a truncated histidine-rich domain (−HHKHH (SEQ ID NO: 46)) resulted in HK polymers that were ineffective carriers of siRNA. That the HK polymer with the truncated histidine rich domain was no more effective than the polymer with the glycine suggest that the buffering capacity of the histidine-rich domain may not be a dominant mechanism for this domain. Moreover, these results indicate that all the domains (the terminal branches and the histidine-rich domain) of the highly branched HK peptides are important for the development of an effective siRNA carrier.

Although the repeating pattern of HHK was present in H³K4b and H³K8b, N-terminal lysine residues were removed in the highly branched polymer, H³K8b. Reduction in the number of lysine residues in the terminal branches of H³K8b may lead to decreased binding of siRNA and increase the amount of siRNA in the cytoplasm compared to that in the nucleus. By adding a single lysine to each terminal branch of H³K8b (eight lysine residues total per polymer), the efficacy of the new polymer ((+K)H³K8b) in reducing the target mRNA was significantly impaired compared to that of H³K8b. A smaller polymer sequence (i.e., those not having the added lysine to each terminal branch) that accomplishes siRNA transport is advantageous in synthesizing polymers more readily. The idea that binding modulates siRNA release is consistent with the finding that a carrier peptide with increased binding to siRNA is less effective as a carrier for siRNA. (Simeoni F, Morris M C, Heitz F, Divita G. Insight into the mechanism of the peptide-based gene delivery system MPG: implications for delivery of siRNA into mammalian cells. Nucleic Acids Res 2003; 31:2717-2724.). Nevertheless, the vast amount of HK carriers with varying abilities to bind nucleic acids were ineffective carriers of siRNA.

H³K8b in complex with siRNA is only smaller in size than the H²K4b/siRNA complex. Varying the HKP/siRNA ratio altered the zeta potential (a measure of a particle surface charge) from positive to negative charge, the transfection activity was minimally effected. In contrast, uptake of the complexes correlated more closely with transfection levels of the polyplexes. HKP-augmented plasmid uptake and protein expression from transfected plasmids significantly more than H³K8b. In contrast, H³K8b siRNA uptake more effectively than other HK polymers or non-viral carriers tested. Although uptake of the nucleic acid by the HK carriers in most cases correlates with the desired effect of the nucleic acid, discrepancies between uptake and the effect of the nucleic acid may occur more often with plasmid-based than with siRNA-delivery systems.

Non-limiting examples of HK polymers according to the present disclosed embodiments include, but are not limited to, one or more polymers selected from the group consisting of H³K8b and (—HHHK)H³K8b. Other modifications may be made by those skilled in the art within the scope of this disclosed embodiments. For example, ligands other than peptides, aptamers, antibodies, carbohydrates such as hyaluronic acid and other ligands that target other receptors, may be added to the polymer(s) within the scope of the present disclosed embodiments. Additionally, polymers in size between and including a HK and (−HHHK)H³K8b polymer are within the scope of the present disclosed embodiments. Further, a fifth or sixth amino acid may be removed from H³K8b and still be within the scope of the present disclosed embodiments.

### Synthesis of Histidine-Lysine Copolymers

Synthesis of histidine-lysine copolymers is well known in the art (see e.g., U.S. Pat. Nos. 7,163,695 and 7,772,201). Briefly, polypeptides may be prepared by any method known in the art for covalently linking any naturally occurring or synthetic amino acid to any naturally occurring or synthetic amino acid in a polypeptide chain which may have a side chain group able to react with the amino or carboxyl group on the amino acids so as to become covalently attached to the polypeptide chain.

For example, but not by way of limitation, branched polypeptides can be prepared as follows: (1) the amino acid to be branched from the main polypeptide chain can be prepared as an N-α-tert-butyloxycarbonyl (Boc) protected amino acid pentafluorophenyl (Opfp) ester and the residue within the main chain to which this branched amino acid will be attached can be an N-Fmoc-2,4-diaminobutyric acid; (2) the coupling of the Boc protected amino acid to diaminobutyric acid can be achieved by adding 5 grams of each precursor to a flask containing 150 ml DMF, along with 2.25 ml pyridine and 50 mg dimethylaminopyridine and allowing the solution to mix for 24 hours; (3) the polypeptide can then be extracted from the 150 ml coupling reaction by mixing the reaction with 400 ml dichloromethane (DCM) and 200 ml 0.12N HCl in a 1 liter separatory funnel, and allowing the phases to separate, saving the bottom aqueous layer and re-extracting the top layer two more times with 200 ml 0.12 N HCl; (4) the solution containing the polypeptide can be dehydrated by adding 2-5 grams magnesium sulfate, filtering out the magnesium sulfate, and evaporating the remaining solution to a volume of about 2-5 ml; (5) the dipolypeptide can then be precipitated by addition of ethyl acetate and then 2 volumes of hexanes and then collected by filtration and washed two times with cold hexanes; and (6) the resulting filtrate can be lyophilized to achieve a light powder form of the desired dipolypeptide. Branched polypeptides prepared by this method will have a substitution of diaminobutyric acid at the amino acid position, which is branched. Branched polypeptides containing an amino acid or amino acid analog substitution other than diaminobutyric acid can be prepared analogously to the procedure described above, using the N-Fmoc coupled form of the amino acid or amino acid analog.

Polypeptides of the transport polymer can also be encoded by viral DNA and be expressed on the virus surface. Alternatively, histidine could be covalently linked to proteins through amide bonds with a water soluble di-carbodimide.

The HK transport polymer may also include a polypeptide—“synthetic monomer” copolymer. In these embodiments, the transport polymer backbone may comprise covalently linked segments of polypeptide and segments of synthetic monomer or synthetic polymer. The synthetic monomer or polymer may be biocompatible and/or biodegradable. Examples of synthetic monomers include ethylenically or acetylenically unsaturated monomers containing at least one reactive site for binding to the polypeptide. Suitable monomers as well as methods for preparing a polypeptide—“synthetic monomer” copolymer are described in U.S. Pat. No. 4,511,478, for “Polymerizable compounds and methods for preparing synthetic polymers that integrally contain polypeptides,” by Nowinski et al, which is herein incorporated by reference. Where the transport polymer comprises a branched polymer, synthetic monomer or polymer may be incorporated into the backbone(s) and/or branch(es). Furthermore, a backbone or branch may include a synthetic monomer or polymer. Finally, in this embodiment, the branching monomers may be branching amino acids or branching synthetic monomers. Branching synthetic monomers may include for example, ethylenically or acetylenically unsaturated monomers containing at least one substituent reactive side-group. Additionally these side groups may consist of peptide (or non-peptide) sequences that are able to bind to select targets on cell membranes—providing the ability to specifically deliver siRNAs or other nucleotides to specific cell types within an organism.

Transport HK polymers in accordance with the present disclosed embodiments may be synthesized by methods known to those skilled in the art. By way of non-limiting example, certain HK polymers discussed herein may be synthesized as follows. The Biopolymer Core Facility at the University of Maryland may be used to synthesize for example, the following HK polymers on a Ranin Voyager solid-phase synthesizer (PTI, Tucson, Ariz., USA): (1) H²K4b (83mer; molecular weight 11137 Da); (2) H³K4b (71mer; MW 9596 Da); (3) HK4b (79mer; MW 10896 Da); (4) H³K8b (163mer; MW 23218 Da); (5) H³K8b (166mer; MW 23564 Da); (6) (−HHHK)H³K8b (131mer; MW 18901 Da); (7) (−HHHK)H³K8b (134mer; MW 19243 Da); (8) ((K+) H³K8b (174mer; MW 24594 Da). The structures of certain branched polymers are shown in FIG. 1. The polymers with four branches (e.g., H³K4b, HK4b) may be synthesized by methods known in the art. The sequence of synthesis for highly branched polymers with eight terminal branches may be as follows: (1) RGD or other ligand (if present); (2) the 3-lysine core; (3) histidine-rich domain; (4) addition of a lysine; and (5) terminal branches. The RGD sequence may be initially synthesized by the instrument followed by three manual couplings with (fmoc)-Lys-(Dde)(the lysine core). The (Dde) protecting groups may be removed during the automatic deprotection cycle. To the lysine core, activated amino acids that comprise the histidine-rich domain may then be added sequentially by the instrument. A (fmoc)-Lys-(fmoc) amino acid was added to the histidine-rich domain and the fmoc protecting groups were then removed. To the .alpha. and .epsilon. amine groups of this lysine, activated amino acids of the terminal branches may then be added. The peptide is cleaved from the resin and precipitated by methods known in the art.

By way of non-limiting example, polymers of the disclosed embodiments may be analyzed as follows. Polymers may be first analyzed by high-performance liquid chromatography (HPLC; Beckman, Fullerton, Calif, USA) and might not be further purified if HPLC reveals that the purity of polymers is 95% or greater. The polymers may be purified on an HPLC column, for example with System Gold operating software, using a Dynamax 21-4.times.250 mm C-18 reversed phase preparative column with a binary solvent system. Detection may be at 214 nm. Further analyses of the polymers may be performed for example, using a Voyager matrix-assisted laser desorptionionization time-of-flight (MALDI-TOF) mass spectrometer (Applied Biosystems, Foster City, Calif., USA) and amino acid analysis (AAA Laboratory Service, Boring, Oreg., USA). Transfection agents such as, SuperFect (Qiagen, Valencia, Calif), Oligofectamine (Invitrogen, Carlsbad, Calif), Lipofectamine 2000 (Invitrogen), and Lipofectamine (Invitrogen) may be used according to the manufacturers' instructions. DOTAP liposomes may be prepared by methods known in the art.

Suitable HKP copolymers are described in WO/2001/047496, WO/2003/090719, and WO/2006/060182. HKP copolymers form a nanoparticle containing an siRNA molecule, typically 100-400 nm in diameter. HKP and HKP(+H) both have a lysine backbone (three lysine residues) where the lysine side chain ε-amino groups and the N-terminus are coupled to [KH₃]₄K (SEQ ID NO: 25) (for HKP) or KH₃KH₄[KH₃]₂K (SEQ ID NO:32) (for HKP(+H). The branched HKP carriers can be synthesized by methods that are well-known in the art including, for example, solid-phase peptide synthesis.

Formation of Nanoparticles Comprising Copolymer and siRNA

Nanoparticles advantageously are formed and included as part of a pharmaceutical composition for administration to a subject. Various methods of nanoparticle formation are well known in the art. See, e.g., Babu et al., IEEE Trans Nanobioscience, 15: 849-863 (2016).

Nanoparticles may be formed using a microfluidic mixer system, in which the pharmaceutical composition comprising one or more siRNA molecules and one or more HKP copolymers are mixed at a fixed or variable flow rate. The flow rate can be varied to modulate the size of the nanoparticles produced, e.g., if the fixed flow rate is producing nanoparticles of a diameter that is too large.

As discussed above, transport polymers, that include histidine (H) and lysine (K) in the disclosed embodiments include one or more carriers that are effective for transporting siRNA, including for example, polymers having between six and 10 terminal branches. According to certain embodiments, the transport polymer of the present disclosed embodiments includes eight terminal branches and a histidine-rich domain. According to certain embodiments, the transport polymer comprises a terminal branch having a sequence of -HHHKHHHKHHHKHHHKHHH- (SEQ ID NO: 44) or a version thereof. Non-limiting examples of transport polymers in accordance with the present disclosed embodiments include one or more polymers selected from H³K8b and structural analogs, including one or more other ligand(s) such as (—HHHK)H³K8b, and the like.

Transport polymers of the present disclosed embodiments may optionally include one or more stabilizing agents. Suitable stabilizing agents would be apparent to those skilled in the art in view of this disclosure. Nonlimiting examples of stabilizing agents in accordance with the present disclosed embodiments include polyethyleneglycol (PEG) or hydroxypropylmethylacrylimide (HPMA).

Transport polymers of the present disclosed embodiments may optionally include one or more targeting ligands. Suitable targeting ligands would be apparent to those skilled in the art in view of this disclosure.

The disclosed embodiments are further directed to compositions, which include transfection complexes of the present disclosed embodiments. Such compositions may include for example, one or more intracellular delivery components in association with the HK polymer and/or the siRNA. The intracellular delivery component may include for example, a lipid (such as cationic lipids), a transition metal or other components that would be apparent to those skilled in the art.

In certain embodiments, the composition comprises a suitable carrier, such as a pharmaceutically acceptable carrier. In these embodiments, there may or may not be a viral or liposomal component. In these embodiments, the complex formed by the transport polymer and the siRNA may be stable at a pH between about 4.0 and 6.6, or up to 7.4, but preferably in the acidic range, below about 6.9.

In certain embodiments, transfection complex compositions include a transport polymer (which may act as an intracellular delivery component) and siRNA. In these embodiments the transport polymer may act as the intracellular delivery component without need for additional delivery components, or may act in conjunction with other delivery components.

In other embodiments, the transfection complex compositions may include (i) the transport polymer, (ii) at least one intracellular delivery component in association with the transport polymer, and (iii) siRNA in association with the intracellular delivery component and/or the transport polymer. Methods of making these compositions may include combining (i) and (ii) for a time sufficient for the transport polymer and the siRNA to associate into a stable complex. Components (i), (ii) and (iii) may also be provided in a suitable carrier, such as a pharmaceutically acceptable carrier. In embodiments that include an intracellular delivery component other than the transport polymer, the transport polymer may interact with an intracellular delivery component, such as a liposome, through non-covalent or covalent interactions. The transport polymer may interact with siRNA through non-covalent or covalent interactions. Alternatively, the transport polymer need not interact directly with the siRNA, but rather, the transport polymer may react with an intracellular delivery component(s), which in turn interacts with the siRNA, in the context of the overall complex.

The present disclosed embodiments further include assays for determining an effective carrier of siRNA for transfection into cells. These assays include mixing siRNA with a transport polymer to form a transfection complex; contacting the transfection complex with one or more cells; and detecting the presence or absence of siRNA activity within the cells. In certain embodiments, the siRNA is directed toward beta-galactosidase.

### Delivery Components

Intracellular delivery components of the presently disclosed embodiments comprise the transport polymer itself. Where intracellular delivery components other than the transport polymer are utilized such delivery components may be viral or non-viral components. Suitable viral intracellular delivery components include, but are not limited to, retroviruses (e.g., murine leukemia virus, avian, lentivirus), adenoviruses and adeno-associated viruses, herpes simplex viruses, rhinovirus, Sendai virus, and Poxviruses. Suitable non-viral intracellular delivery components include, but are not limited to, lipids and various lipid-based substances, such as liposomes and micelles, as well as various polymers known in the art.

Suitable lipids include, but are not limited to, phosphoglycerides, sphingolipids, phosphatidylcholine, phosphatidylethanolamine, phosphatidylserine, phosphatidylinositol, phosphatidic acid, palmitoyloleoyl phosphatidyleholine, lysophosphatidylcholine, lysophosphatidylethanolamine, dipalmitoylphosphatidylcholine, dioleoylphosphatidylcholine, distearoylphosphatidylcholine, dilinoleoylphosphatidylcholine, glycosphingolipid, amphipathic lipids. The lipids may be in the form of unilamellar or multilamellar liposomes.

The intracellular delivery component may include, but are not limited to, a cationic lipid. Many such cationic lipids are known in the art. A variety of cationic lipids have been made in which a diacylglycerol or cholesterol hydrophobic moiety is linked to a cationic headgroup by metabolically degradable ester bond, for example: 1,2-Bis(oleoyloxy)-3-(4-′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC) and cholesteryl (4′-trimethylammonio)butanoate (ChoTB). Other suitable lipids include, but are not limited to, cationic, non-pH sensitive lipids, such as: 1,2-dioleoyl-3-dimethyl-hydroxyethyl ammonium bromide (DORI), 1,2-dioleyloxypropyl-3-dimethyl-hydroxyethyl ammonium bromide (DORIE), and 1,2-dimyristyloxypropyl-3-dimethyl-hydroxyethyl ammonium bromide (DMRIE). Other non-pH-sensitive, cationic lipids include, but are not limited to: O,O′-didodecyl-N-[p-(2-trimethylammonioethyloxy)benzoyl]-N,N,N-trimethylammonium chloride, Lipospermine, DC-Chol (3 beta [N—(N′,N″-dimethylaminoethane) carbonyl]cholesterol), lipopoly(L-lysine), cationic multilamellar liposomes containing N-(alpha-trimethylammonioacetyl)-didodecyl-D-glutamate chloride (TMAG), TransfectACE™ (1:2.5 (w:w) ratio of DDAB which is dimethyl dioctadecylammonium bromide and DOPE) (Invitrogen) and lipofectAMINE™ (3:1 (w:w) ratio of DOSPA which is 2,3-dioleyloxy-N-[20([2,5-bis[(3-amino-propyl)amino]-1-oxypentyl]amino)et-hyl]-N,N-dimethyl-2,3-bis(9-octadecenylo-xy)-1-propanaminium trifluoroacetate and DOPE) (Invitrogen). Other suitable lipids are described in U.S. Pat. No. 5,965,434, for “Amphipathic PH sensitive compounds and delivery systems for delivering biologically active compounds,” by Wolff et al.

Cationic lipids that may be used in accordance with the presently disclosed embodiments comprise, but are not limited to, those that form liposomes in a physiologically compatible environment. Suitable cationic lipids include, but are not limited to cationic lipids selected from the group consisting of 1,2-dioleythyloxypropyl-3-trimethyl ammonium bromide; 1,2-dimyristyloxypropyl-3-dimethyl-hydroxyethyl ammonium bromide; dimethyldioctadecyl ammonium bromide; 1,2-dioleoyl-3-(trimethylammonium)propane (DOTAP); 3.beta.N—(N′,N′-dimethylaminoethane)carbamoyl]cholesterol (DC-cholesterol); 1,2 dioleolyl-sn-glycero-3-ethylphosphocholine; 1,2 dimyristoyl-sn-glycero-3-ethylphosphocholine; [1-(2,3-diol-eyloxy)propyl]-N,N,N-trimethyl-ammonium chloride (DOTMA); 1,3-dioleoyloxy-2-(6carhoxys-permyl) propylamide (DOSPER); 2,3-dioleyloxy-N-[2(spermine-carboxyamido)ethyl]-N,N, dimethyl-1-propanamoniumtrifluoroacetate (DOSPA); and 1,2-dimyristyloxypropyl-3-dimethyl-hydroxyethyl ammonium bromide (DMRIE).

Cationic lipids may be used with one or more helper lipids such as diloleoylphosphatidylethanolamine (DOPE) or cholesterol to enhance transfection. The molar percentages of these helper lipids in cationic liposomes are between about 5 and 50%. In addition, pegylated lipids, which can prolong the in vivo half-life of cationic liposomes, can be present in molar percentages of between about 0.05 and 0.5%.

Compositions in accordance with the disclosed embodiments may alternatively include one or more components to enhance transfection, to preserve reagents, or to enhance stability of the delivery complex. For example, in certain embodiments stabilizing compounds such as polyethylene glycol can be covalently attached to either the lipids or to the transport polymer.

Compositions of the disclosed embodiments may also suitably comprise various delivery-enhancing components known in the art. For example, the composition may comprise one or more compounds known to enter the nucleus or ligands subject to receptor-mediated endocytosis, and the like. For example, the ligand may comprise a fusogenic viral peptide to disrupt endosomes, allowing the nucleic acid to avoid lysosomal degradation. Other examples of delivery-enhancing components include, but are not limited to, nuclear proteins, adenoviral particles, transferrin, surfactant-B, anti-thrombomodulin, intercalating agents, hemagglutinin, asialoglycoprotein, chloroquine, colchicine, integrin ligands, LDL receptor ligands, and viral proteins to maintain expression (e.g., integrase, LTR elements, rep proteins, oriP and EBNA-1 proteins) or viral components that interact with the cell surface proteins (e.g., ICAM, HA-1, MLV's gp70-phosphate transporter, and HIV's gp120-CD4). Delivery enhancing components can be covalently or non-covalently associated with the transport polymer, the intracellular delivery component, or the pharmaceutical agent. For instance, delivery to a tumor vasculature can be targeted by covalently attaching a -RGD- or -NGR- motif. This could be accomplished using a peptide synthesizer or by coupling to amino groups or carboxyl groups on the transport polymer with a water-soluble di-carbodiimide (e.g., 1-ethyl-3-(3-dimethylaminopropyl)carbodiimide). Both of these methods are known to those familiar with the art.

Compositions of the present disclosed embodiments may suitably include a transition metal ion, such as a zinc ion. The presence of a transition metal in the complexes of the disclosed embodiments may enhance transfection efficiency.

### Administration

Delivery of these siRNAs to the tumor environment within the animal/human exhibiting the disease can be accomplished using a variety of targeted or non-targeted delivery agents as components of a pharmaceutical composition. These delivery vehicles comprise lipids, modified lipids, peptide delivery vehicles and the like or can even be via direct attachment of a targeting ligand onto a modified (chemically stable) siRNA molecule through modification of the backbone to prevent degradation of the siRNA by nucleases and other enzymes encountered in the circulation.

The pharmaceutical compositions described herein may be administered to subjects, including human subjects, by any mode of administration that is conventionally used to administer compositions. Thus, the compositions can be in the form of an aerosol, dispersion, solution, or suspension and can be formulated for inhalation, intramuscular, oral, sublingual, buccal, parenteral, nasal, subcutaneous, intradermal, or topical administration. The term parenteral as used herein includes percutaneous, subcutaneous, intravascular (e.g., intravenous), intramuscular, or intrathecal injection or infusion techniques and the like.

As used herein, an effective dose of a composition is the dose required to produce a protective immune response in the subject to whom the pharmaceutical composition is administered. A protective immune response in the present context is one that prevents or ameliorates a variety of diseases or disorders.

The composition may be administered one or more times. An initial measurement of an desired effect to the composition may be made by measuring one or more compounds in the circulation or tissue samples of the recipient subject. Methods of measuring a variety of compounds in this manner are also well known in the art, as is an appropriate dose effective in preventing or inhibiting the occurrence, or treating (alleviate a symptom to some extent, preferably all of the symptoms) of a disease state.

The pharmaceutically effective dose depends on the type of disease, the composition used, the route of administration, the type of mammal being treated, the physical characteristics of the specific mammal under consideration, concurrent medication, and other factors that those skilled in the medical arts will recognize that, generally, an amount between 0.1 mg/kg and 100 mg/kg body weight/day of active ingredients is administered dependent upon potency of the formulated composition, between about 0.1 mg/kg and about 1.0 mg/kg, between about 1.0 mg/kg and about 2.0 mg/kg, from between about 2.0 mg/kg and 3.0 mg/kg, between about 3.0 and 5.0 mg/kg, between about 5 mg/kg and about 8 mg/kg, between about 8 mg/kg and about 15 mg/kg, between about 15 mg/kg and about 25 mg/kg, between about 25 mg/kg and about 35 mg/kg, between about 35 mg/kg and about 45 mg/kg, between about 45 mg/kg and about 55 mg/kg, between about 55 mg/kg and about 65 mg/kg, between about 65 mg/kg and about 75 mg/kg, between about 75 mg/kg and about 85 mg/kg, between about 85 mg/kg and about 95 mg/kg, and between about 95 mg/kg and about 105 mg/kg.

Their application, however, has until recently been restricted by the instability and inefficient in vivo delivery of nucleic acids such as siRNA molecules. The methods described herein provide methods of making and using pharmaceutical compositions with a HK copolymer nanoparticle delivery system.

The methods described herein may be used in clinical applications of the siRNA include prophylactic and therapeutic compositions effective against various diseases, especially infectious diseases and oncology indications.

### Treatment of Subjects

The present disclosed embodiments also provide methods of treating viral diseases comprising using the complexes or compositions of the present disclosed embodiments. In particular, methods are provided for treating a subject—human or other—having a disease or disorder, by administering to the subject a therapeutically effective amount of a complex or composition of the present disclosed embodiments. Also encompassed are methods for treating a subject having a disease, by administering to the subject cells that have been transfected by the methods disclosed herein. Examples of genetic and/or non-neoplastic diseases potentially treatable with the complex, compositions, and methods include, but are not limited to the following: adenosine deaminase deficiency; purine nucleoside phosphorylase deficiency; chronic granulomatous disease with defective p47phox; sickle cell with HbS, β-thalassemia; Faconi's anemia; familial hypercholesterolemia; phenylketonuria; ornithine transcarbamylase deficiency; apolipoprotein E deficiency; hemophilia A and B; muscular dystrophy; cystic fibrosis; Parkinsons, retinitis pigmentosa, lysosomal storage disease (e.g., mucopolysaccharide type 1, Hunter, Hurler and Gaucher), diabetic retinopathy, human immunodeficiency virus disease, virus (e.g., HPV, HBV) infection, acquired anemia, cardiac and peripheral vascular disease, and arthritis. In some of these examples of diseases, the therapeutic gene may encode a replacement enzyme or protein of the genetic or acquired disease, an antisense or ribozyme molecule, a decoy molecule, or a suicide gene product.

The present disclosed embodiments also disclose a method of ex vivo gene therapy comprising: (i) removing a cell from a subject; (ii) delivering a nucleic acid (such as siRNA) to the interior of the cell by contacting the cell with a transfection complex or composition comprising such a transfection complex of the present disclosed embodiments; and (iii) administering the cell comprising the nucleic acid (e.g., siRNA) to the subject.

Recombinant cells may be produced using the complexes of the present disclosed embodiments. Resulting recombinant cells can be delivered to a subject by various methods known in the art. In certain embodiments, the recombinant cells are injected, e.g., subcutaneously. In other embodiments, recombinant skin cells may be applied as a skin graft onto a human subject, for example. Recombinant blood cells (e.g., hematopoietic stem or progenitor cells) are preferably administered intravenously. The cells can also be encapsulated in a suitable vehicle and then implanted in the subject. The amount of cells administered depends on a variety of factors known in the art, for example, the desired effect, subject state, rate of expression of the chimeric polypeptides, etc., and can readily be determined by one skilled in the art.

All ranges and ratios disclosed here can and necessarily do describe all subranges and subratios therein for all purposes, and all such subranges and subratios also form part and parcel of the disclosed embodiments. Any listed range or ratio can be easily recognized as sufficiently describing and enabling the same range or ratio being broken down into at least equal halves, thirds, quarters, fifths, tenths, etc. As a non-limiting example, each range or ratio discussed herein can be readily broken down into a lower third, middle third and upper third, etc.

The embodiments disclosed of pharmaceutical formulations may be used alone or in combination with other treatments or components of treatments for other dermatological or nondermatological disorders.

The disclosed embodiments will be better understood by reference to the following examples, which are intended for purposes of illustration and are not intended to be interpreted in any way to limit the scope of the appended claims.

The word “exemplary” is used herein to mean “serving as an example, instance, or illustration.” Any embodiment described herein as “exemplary” is not necessarily to be construed as preferred or advantageous over other embodiments.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 ¶6. It will be apparently to those having skill in the art that changes may be made to the details of the above-described embodiments without departing from the underlying principles of the disclosed embodiments.

While specific embodiments and application of the disclosed embodiments have been illustrated and described, the disclosed embodiments are not limited to the precise configuration and components disclosed herein. Various modifications, changes, and variations, which will be apparent to those skilled in the art may be made in the arrangement, operation, and details of the methods and systems of the embodiments disclosed herein, including those of the appended claims. Finally, various features of the disclosed embodiments herein may be combined to provide additional configurations, which fall within the scope of the disclosed embodiments. The following examples illustrate the kinetic measures and the efficacy of inhibitory compounds tested, including those in the disclosed embodiments.

1. An oligonucleotide molecule comprising: nucleotides and one or more antiviral nucleoside analogs.

2. The oligonucleotide molecule of claim 1, wherein the nucleotides comprise deoxyribonucleotides.

3. The oligonucleotide molecule of claim 1, wherein the nucleotides comprise ribonucleotides.

4. The oligonucleotide molecule of claim 1, wherein the nucleotides comprise deoxyribonucleotides and ribonucleotides.

5. The oligonucleotide molecule of claim 1, wherein the one or more antiviral nucleoside analogs are attached to one end of the molecule.

6. The oligonucleotide of claim 1, wherein the one or more antiviral nucleoside analogs are within the molecule.

7. The oligonucleotide of claim 1, wherein said nucleoside analog is selected from the group consisting of Abacavir, Acyclovir, Adefovir, Cidofovir, Clevudine, cytarabine, Didanosine, didanosine (ddI), emtricitabine, Emtricitabine, Entecavir, Famciclovir, galidesivir, Ganciclovir/Valganciclovir, gemcitabine, GS-441524, idoxuridine, lamivudine (3TC), Molnupiravir, Remdesivir, Ribavirin, Sofosbuvir, stavudine (d4T), Telbivudine, Tenofovir, trifluridine, Valacyclovir, vidarabine, zalcitabine (ddC), and Zidovudine.

8. An siRNA duplex comprising an oligonucleotide according to claim 1.

9. A pharmaceutical composition comprising: an oligonucleotide according to claim 1.

10. The pharmaceutical composition according to claim 9, further comprising a histidine-lysine copolymer.

11. A method of treating a viral infection in a subject, comprising: administering to the subject an effective amount of a pharmaceutical composition according to claim 9.
